(19) United States
(12) Patent Application Publication
(10) Pub. No.: US 2026/0183222 A1
(43) Pub. Date: Jul. 2, 2026

(54) USE OF A HALOPTERIS SCOPARIA EXTRACT

(71) Applicant: SOCIETE D'EXPLOITATION DE PRODUITS POUR LES INDUSTRIES CHIMIQUES SEPPIC, Paris (FR)

(72) Inventors: Cecile Bize, Paris (FR); Catherine Kern, Paris (FR); Sophie Pecastaings, Paris (FR); Lise Laurencon, Paris (FR)

(73) Assignee: SOCIETE D'EXPLOITATION DE PRODUITS POUR LES INDUSTRIES CHIMIQUES SEPPIC, Paris (FR)

(21) Appl. No.: 19/439,216

(22) Filed: Jan. 2, 2026

(30) Foreign Application Priority Data
Jan. 2, 2025 (FR) 2500014

(51) Int. Cl. A61K 8/9711; A61K 36/03; A61P 17/08; A61Q 19/00

(52) U.S. Cl. CPC A61K 8/9711; A61K 36/03; A61P 17/08; A61Q 19/008; A61K 2236/15; A61K 2236/17; A61K 2236/333; A61K 2236/53

(57) ABSTRACT

A method for reducing the amount of sebum produced by human skin and/or by the scalp, for preventing, reducing, slowing down, stopping, and/or attenuating the appearance of unsightly, uncomfortable, and/or unpleasant manifestations of the skin and/or scalp in humans due to the secretion of sebum includes topically applying an extract of the alga Halopteris scoparia. Cosmetic or pharmaceutical compositions, in particular dermatological compositions, for topical use, containing an extract of the alga Halopteris scoparia, are also described.

### TECHNICAL FIELD

The present invention relates to the use of an extract of the alga Halopteris scoparia for reducing the amount of sebum produced by human skin and/or scalp, and/or for preventing and/or reducing and/or slowing down and/or stopping and/or attenuating the presence of closed comedones and/or open comedones and/or papules and/or pustules and/or nodules and/or scars and/or visibly dilated pores and/or a glistening or greasy or shiny appearance and/or the tacky sensation of the skin and/or scalp in humans due to secretion of sebum, in particular in cosmetic or pharmaceutical compositions, in particular dermatological compositions, for topical use.

### BACKGROUND

Human skin constitutes the first visual image offered for others to see and, consequently, improving its appearance is often a subject of constant concern for human beings. The skin is a reflection either of a state of well-being, often associated with clear or radiant skin, or, conversely, of a state of tiredness and/or negligence, often associated with greasy or glistening skin.

The skin is an atypical organ of the human body, which is extremely thin with regard to its extent, but is also an individual's heaviest organ. One of the characteristics of the skin lies in the fact that it is an interface organ, a boundary organ, between the internal medium (human body) and the external environment. As a result, and with the flora which covers it and lives thereon, the skin is the first barrier for protecting the human body.

Due to its interface position with the external environment, the skin is subjected to numerous daily stresses, for instance contact with clothing, changes in temperature, changes in humidity levels, changes in pressure, or even to attacking factors, for instance contact with certain chemicals which have or may have a very acidic, or very basic, or irritant nature, with chemicals regarded as polluting agents.

### DESCRIPTION

The skin is composed of layers of different tissues:
the epidermis, composed of keratinocytes, is its outermost part, followed by
the dermis, which is a connective tissue mainly composed of fibroblasts and of extracellular matrix, and
the hypodermis, consisting of adipocytes, which is the deepest part and the part furthest from the external environment.

The skin performs various functions in the interest of the entire system which it shelters, among which are included the following:
a mechanical barrier function in order to guarantee the integrity of the internal medium of the body,
an emunctorial function directed towards secreting sweat based on water, salts and acidic waste,
a function of regulating the temperature of the body, and contains many other regulatory mechanisms, for instance its mechanism of adaptation and of protection against ultraviolet radiation (adaptive pigment colouring by the production of melanin), for instance a system for immune monitoring by the presence of macrophages or dendritic cells.

Human skin also constitutes the first image offered for others to see. Consequently, improving its appearance is a matter of constant concern for humans. The skin is the reflection of a state of well-being, often associated with youthfulness, and, conversely, with a state of fatigue and/or ageing. As a result, preserving and improving the state of the outermost layer of the skin, namely the epidermis, is a major focus for the research conducted by the cosmetics industries. Thus, even essentially healthy skin, that is to say skin that regulates itself well most of the time, that is neither too greasy nor too dry, remains a living organ that can react to internal or external disturbances.

At the periphery of the epidermis is an upper cornified layer known as the stratum corneum, which is the first layer of the epidermis to suffer the stresses of external origin, such as variations in external climatic conditions (temperature, pressure, hygrometry) or mechanical stresses.

The stratum corneum is more particularly in contact with the skin microbiota.

For the purposes of the present patent application, the term “skin microbiota” denotes a population of specialized or opportunistic microorganisms, for instance bacteria, fungi, yeasts, and the like, which live on the surface of the skin.

The skin microbiota cannot be defined in a specific and generalized manner for all individuals. Since the launch in 2007 of the National Institute of Health's “Human Microbiome Project” (HMP), researchers have been able to observe large topographical variations in the human microbiota and also large differences between individuals.

At least nineteen phyla have been identified, the four main ones of which are Actinobacteria (51.8%), Firmicutes (24.4%), Proteobacteria (16.5%) and Bacteroidetes (6.3%). The genera predominantly identified are Corynebacterium, Propionibacterium and Staphylococcus. The abundance of each group is strongly dependent on the different locations. The fungal organisms isolated from the skin are of the genus Malassezia spp. Furthermore, mites of the genus Demodex are also present and reside in the pilosebaceous units, most often of the surface of the face.

This microbiota feeds both on molecules excreted by the skin (lipids, proteins, etc.) and on compounds secreted by communities of microorganisms, demonstrating real interaction within this microbiota. In addition, this relationship with the host constitutes a true symbiosis.

Bacteria can be commensal when they live in contact with the cutaneo-mucous covering of a host without causing damage. A balance is then established between the individual and the various commensal flora of the skin and mucous membranes, but this balance is constantly threatened by the physical or chemical attacks undergone by the stratum corneum, for instance pollution, variations in temperature, ultraviolet radiation, the intensive use of detergent surfactants, stress, etc. Alongside these commensal bacteria are unwanted and/or pathogenic opportunistic bacteria.

Staphylococcus epidermidis (S. epidermidis) constitutes more than 90% of the resident aerobic flora present in the stratum corneum. The resident flora is also composed of anaerobic bacteria belonging to the division of the Actinobacteria, such as Propionibacterium acnes (P. acnes), frequently found in sebaceous regions, for instance the back, the face and the scalp.

While the normal flora of the skin constitutes a defense for the host, an increase or a reduction in the bacterial composition (dysbiosis) leads to skin inflammation and can be the cause of the development of certain skin pathologies, such as acne, atopic dermatitis or even psoriasis.

Greasy skin is linked to a biological phenomenon called hyperseborrhea or sebaceous hypersecretion, defined as being an abnormally high production of sebum by the sebaceous gland of the skin (sebum hyperproduction). This sebum, flowing on the surface of the skin, thus gives it that unsightly “glistening” feature. This hyperseborrhea is generally associated with an excess presence of the hormone testosterone which, once in the sebaceous gland, is converted by 5α-reductase into dihydrotestosterone (DHT), which binds to the receptors present in sebaceous cells and activates the synthesis of sebum.

Hyperseborrhea is accompanied by follicular hyperkeratinization, characterized by an increase in the number of keratinocytes in the follicular infundibulum, which causes an obstruction of the follicular canal and makes it difficult for the sebum to flow.

This results in an accumulation of sebum which is beneficial to the growth of the anaerobic bacterium Propionibacterium acnes. The latter will bring about the appearance of an inflammatory phenomenon. These phenomena together result in the appearance of characteristic clinical signs, such as comedones, characterizing acne-prone skin.

The presence of the bacterium P. acnes in the pilosebaceous structure is known to be involved in the development of the pathology. Acne vulgaris is a skin disease which is linked to dysbiosis, i.e. an imbalance in the skin microbiota. Excessive consumption of tobacco and alcohol also appear to be among the factors that may increase the effects of acne vulgaris. Among other environmental factors, stress, urban pollution ans also the sun (UV radiation inducing the peroxidation of sebum and in particular of squalene), are also recognized as being involved in the worsening of the symptoms of acne vulgaris.

The colonization of the pilosebaceous follicle by P. acnes is an important factor for the inflammatory reaction in acne vulgaris. In fact, acne is not in the strict sense an infectious disease, since this germ first exerts an inflammatory action, linked to its very numerous enzymatic and chemical secretions and to the immunological reactions it brings about. Thus, P. acnes stimulates the production by the sebocytes, the keratinocytes and the leukocytes (lymphocytes and monocytes) of numerous inflammatory cytokines (IL-1α, IL1β, IL-6, IL-8, IL-10, IL-12, IL-17, IL-18, TNF-α, GM-CSF, and IFN-γ) and also of antimicrobial peptides (defensins and cathelicidins), matrix metalloproteinases, reactive oxygen species, and other products involved in the inflammatory response. In addition, P. acnes secretes a lipase which hydrolyses the triglycerides of sebum to give free fatty acids which are irritant and chemotactic to neutrophils.

Among the plant extracts that may be used for their actions on human microbiota, mention may be made of a lyophilized extract of burdock (or Arctium lappa) leaves for which antibacterial activity has been demonstrated and more particularly activity against oral microorganisms, appearing more effective against bacteria associated with endodontic pathogens, such as Bacillus subtilis, Candida albicans, Lactobacillus acidophilus and Pseudomonas aeruginosa.

Halopteris scoparia is a brown alga, of the order Sphacelariale migula, of the family Stypocaulaceae, of the genus Halopteris, of the species scoparia. It comes in the form of highly branched tufts 10 to 20 centimetres high.

As part of their research into new active principles for preventing and/or treating the appearance of unsightly and/or uncomfortable and/or unpleasant manifestations of the skin and/or scalp, the inventors focused on developing a new technical solution based on an extract of the alga Halopteris scoparia to have effects of decreasing or regulating sebum, in particular excess sebum, on the skin and/or scalp in humans, in particular dermopurifying or seboregulatory effects.

According to a first aspect, a subject of the invention is the topical cosmetic use of an extract of Halopteris scoparia as a cosmetic active principle for preventing and/or reducing and/or slowing down and/or stopping and/or attenuating the presence of closed comedones and/or open comedones and/or papules and/or pustules and/or nodules and/or scars and/or visibly dilated pores and/or the glistening or greasy or shiny appearance and/or the tacky sensation due to secretion of sebum from essentially healthy skin.

According to a particular aspect of the present invention, the extract of Halopteris scoparia comprises, per 100% of its mass:
from 80% by mass to 95% by mass of water, preferably from 85% by mass to 90% by mass and even more preferentially from 87% by mass to 89% by mass of water;
from 4.5% by mass to 15% by mass, preferably from 6% by mass to 12% by mass, even more preferentially from 8% by mass to 10% by mass of an organic solvent chosen from the group consisting of 1,2-propanediol, 1,3-propanediol, 1,4-butanediol, 1,3-butanediol, 1,2-butanediol, 1,2-pentanediol, 2-methyl-2,4-pentanediol, 1,6-hexanediol, 1,8-octanediol, and a mixture of these compounds;
from 0.1% by mass to 5% by mass, preferably from 0.5% by mass to 4% by mass, more preferentially from 1% by mass to 3% by mass, even more preferentially from 1.2% by mass to 1.5% by mass of dry matter.

According to a particular aspect of the present invention, the organic solvent present in the extract of Halopteris scoparia may be a mixture of 1,3-propanediol and 1,2-pentanediol.

According to a particular aspect of the present invention, the organic solvent may comprise, per 100% of its mass, a mixture of two diols different from each other in equal mass proportions, i.e. 50% by mass of a first diol and 50% by mass of a second diol different from the first diol.

According to a particular aspect of the present invention, the extract of Halopteris scoparia can be obtained via a process comprising the following successive steps:
a step a) of drying and grinding the alga Halopteris scoparia to obtain a biomass;
a step b) of bringing the biomass obtained in step a) into contact with said organic solvent so as to obtain a mixture, said mixture comprising, per 100% of its mass, from 5% by mass to 15% by mass of the biomass and from 85% by mass to 95% by mass of said organic solvent;
a step c) of stirring the mixture obtained in step b) for a period of between 1 hour and 6 hours at a temperature of between 15° C. and 35° C.;
a step d) of separating the biomass from the mixture obtained at the end of step c) in order to isolate a liquid phase which corresponds to the extract of Halopteris scoparia.

According to a particular aspect of the present invention, the unsightly and/or uncomfortable and/or unpleasant manifestations of the skin and/or scalp in humans due to the secretion of sebum are chosen from the presence of closed comedones (whiteheads or microcysts) and/or open comedones (blackheads) and/or papules and/or pustules and/or nodules and/or scars and/or visibly dilated pores and/or the glistening or greasy or shiny appearance and/or the tacky sensation.

For the purposes of the present invention, the term “unsightly and/or uncomfortable and/or unpleasant manifestations of the skin and/or scalp due to the secretion of sebum” means any modification of the external appearance of the skin and/or scalp, for instance visible signs of changes in the external appearance and/or uncomfortable and/or unpleasant sensations of the skin and/or scalp due to secretion or production of sebum, in particular due to the presence of excess sebum on the skin and/or scalp, for example the presence of closed comedones (whiteheads or microcysts) and/or open comedones (blackheads) and/or papules and/or pustules and/or nodules and/or scars and/or visibly dilated pores and/or the glistening or greasy or shiny appearance and/or the tacky sensation.

For the purposes of the present invention, the term “excess sebum on the skin or the scalp” means a sebum content on the skin of greater than 95 μgrams·cm⁻² or more particularly greater than 120 μgrams·cm⁻², measured with a sebumeter.

For the purposes of the present invention, the term “dermopurifying agent” means a chemical substance or a chemical composition or an extract derived from the biomass of a plant whose property or technical function consists in preventing and/or reducing and/or slowing down and/or stopping and/or attenuating the appearance of unsightly and/or uncomfortable and/or unpleasant manifestations of skin and/or scalp in humans due to the secretion or production of sebum.

According to a particular aspect of the present invention, the extract of Halopteris scoparia is present in a cosmetic composition intended for topical application, also comprising at least one cosmetically acceptable ingredient.

For the purposes of the present invention, “seboregulator” means a chemical substance or a chemical composition or an extract of plant biomass which is capable of participating in the regulation of the activity of the sebaceous glands by the prevention and/or direct reduction of sebum secretion, for instance zinc gluconate and azelaic acid.

According to a particular aspect of the present invention, the extract of Halopteris scoparia is present as an active principle in said cosmetic composition for topical use, in a cosmetically effective amount, in particular in a content of from 0.01% by mass to 5.00% by mass, or from 0.1% by mass to 1% by mass, relative to the total weight of the composition.

A subject of the present invention is also a cosmetic composition for topical use comprising, as active principle, a cosmetically effective amount of an extract of Halopteris scoparia and at least one cosmetically acceptable ingredient. In particular, said cosmetic composition comprises, per 100% of its mass, from 0.01% by mass to 5.00% by mass, or from 0.1% by mass to 1% by mass, in particular 1% by mass, of the extract of Halopteris scoparia as defined above.

The expression “for topical use” used in the definition of the cosmetic composition which is the subject of the present invention means that said composition is in a form suitable for topical administration, formulated to allow its application to the skin, in particular the skin and the scalp, whether it is a direct or indirect application when said composition is integrated, for example in the case of a bodycare product in the form of a textile or paper wipe or sanitary products intended to be in contact with the skin, in particular the skin and scalp.

The term “cosmetically effective amount” of the extract of the alga Halopteris scoparia means an amount which enables the skin and scalp in humans to be protected against the appearance of unsightly and/or uncomfortable and/or unpleasant manifestations of the skin and/or scalp in humans due to the secretion of sebum, in particular chosen from the presence of closed comedones (whiteheads or microcysts) and/or open comedones (blackheads) and/or papules and/or pustules and/or nodules and/or scars and/or visibly dilated pores and/or the glistening or greasy or shiny appearance and/or the tacky sensation.

According to a more particular aspect of the invention, the cosmetic composition which is the subject of the present invention comprises, per 100% of its mass:
from 0.01% to 5.00% by mass of the extract of Halopteris scoparia as defined above, and
from 95.00% to 99.99% by mass of at least one cosmetically acceptable ingredient.

According to a more particular aspect of the invention, the cosmetic composition which is the subject of the present invention comprises, per 100% of its mass:
from 0.03% to 4.00% by mass of the extract of Halopteris scoparia as defined above, and
from 96.00% to 99.97% by mass of at least one cosmetically acceptable ingredient.

According to a more particular aspect of the invention, the cosmetic composition which is the subject of the present invention comprises, per 100% of its mass:
from 0.05% to 3.00% by mass of the extract of Halopteris scoparia as defined above, and
from 97.00% to 99.95% by mass of at least one cosmetically acceptable ingredient.

According to a more particular aspect of the invention, the cosmetic composition which is the subject of the present invention comprises, per 100% of its mass:
from 0.10% to 2.50% by mass of the extract of Halopteris scoparia as defined above, and
from 97.50% to 99.90% by mass of at least one cosmetically acceptable ingredient.

According to a more particular aspect of the invention, the cosmetic composition which is the subject of the present invention comprises, per 100% of its mass:
from 0.30% to 2.00% by mass of the extract of Halopteris scoparia as defined above, and
from 98.00% to 99.70% by mass of at least one cosmetically acceptable ingredient.

According to a more particular aspect of the invention, the cosmetic composition which is the subject of the present invention comprises, per 100% of its mass:
from 0.50% to 1.50% by mass of the extract of Halopteris scoparia as defined above, and
from 98.50% to 99.50% by mass of at least one cosmetically acceptable ingredient.

According to a more particular aspect of the invention, the cosmetic composition which is the subject of the present invention comprises, per 100% of its mass:
from 0.90% to 0.99% by mass of the extract of Halopteris scoparia as defined above, and
from 99.01% to 99.10% by mass of at least one cosmetically acceptable ingredient.

According to a more particular aspect of the invention, the cosmetic composition which is the subject of the present invention comprises, per 100% of its mass:
1% by mass of the extract of Halopteris scoparia as defined above, and
99% by mass of at least one cosmetically acceptable ingredient.

The cosmetic composition comprising the extract of Halopteris scoparia according to the invention is intended in particular for preventing and/or reducing and/or slowing down and/or stopping and/or attenuating the appearance of unsightly and/or uncomfortable and/or unpleasant manifestations of the skin and/or scalp in humans due to the secretion of sebum, in particular chosen from the presence of closed comedones (whiteheads or microcysts) and/or open comedones (blackheads) and/or papules and/or pustules and/or nodules and/or scars and/or visibly dilated pores and/or glistening or greasy or shiny appearance and/or the tacky sensation.

The cosmetic composition may be in the form of an aqueous or aqueous-alcoholic or aqueous-glycolic solution, in the form of a suspension, an emulsion, a microemulsion or a nanoemulsion, whether of water-in-oil, oil-in-water, water-in-oil-in-water or oil-in-water-in-oil type, or in the form of a powder.

The cosmetic composition that is the subject of the present invention may be packaged in a bottle, in a device of “pump-bottle” type, in pressurized form in an aerosol device, in a device equipped with a perforated wall, such as a grill, or in a device equipped with a ball applicator (known as a “roll-on”).

In the context of the invention, a “cosmetically acceptable ingredient” is a chemical and/or biological additive usually used in the field of cosmetic formulations for topical use.

In general, the extract of Halopteris scoparia that is the subject of the present invention may be combined with chemical and/or biological additives usually used in the field of formulations for topical use, such as foaming and/or detergent surfactants, thickening and/or gelling surfactants, thickeners and/or gelling agents, stabilizers, film-forming compounds, solvents and cosolvents, hydrotropic agents, spring or mineral waters, plasticizers, emulsifiers and coemulsifiers, opacifiers, nacreous agents, superfatting agents, sequestrants, chelating agents, oils, waxes, antioxidants, fragrances, essential oils, preserving agents, conditioning agents, deodorants, whitening agents intended for bleaching bodily hairs and the skin, active principles intended to provide a treating and/or protective action with respect to the skin or the scalp or the hair, sunscreens, mineral fillers or pigments, particles which provide a visual effect or which are intended for encapsulating active agents, exfoliating particles, texturing agents, optical brighteners, insect repellents or probiotics.

As examples of foaming and/or detergent surfactants that may be combined with the extract of Halopteris scoparia in the cosmetic composition that is a subject of the present invention as defined previously, mention may be made of anionic, cationic, amphoteric or nonionic foaming and/or detergent surfactants.

Among the anionic foaming and/or detergent surfactants that may be combined with the extract of Halopteris scoparia in the cosmetic composition that is a subject of the present invention as defined previously, mention may be made of alkali metal salts, alkaline-earth metal salts, ammonium salts, amine salts, amino alcohol salts, alkyl ether sulfate salts, alkyl sulfate salts, alkylamido ether sulfate salts, alkylaryl polyether sulfate salts, monoglyceride sulfate salts, α-olefin sulfonate salts, paraffin sulfonate salts, alkyl phosphate salts, alkyl ether phosphate salts, alkyl sulfonate salts, alkylamide sulfonate salts, alkylaryl sulfonate salts, alkyl carboxylate salts, alkyl sulfosuccinate salts, alkyl ether sulfosuccinate salts, alkylamide sulfosuccinate salts, alkyl sulfoacetate salts, alkyl sarcosinate salts, acyl isethionate salts, N-acyl taurate salts, acyl lactylate salts, salts of N-acylamino acid derivatives, salts of N-acyl peptide derivatives, salts of N-acyl protein derivatives or salts of N-acyl fatty acid derivatives.

Among the amphoteric foaming and/or detergent surfactants that may be combined with the extract of Halopteris scoparia in the cosmetic composition that is a subject of the present invention as defined previously, mention may be made of alkylbetaines, alkylamidobetaines, sultaines, alkylamidoalkylsulfobetaines, imidazoline derivatives, phosphobetaines, amphopolyacetates and amphopropionates.

Among the cationic foaming and/or detergent surfactants that may be combined with the extract of Halopteris scoparia in the cosmetic composition that is a subject of the present invention as defined previously, mention may be made in particular of quaternary ammonium derivatives.

Among the nonionic foaming and/or detergent surfactants that may be combined with the extract of Halopteris scoparia in the cosmetic composition that is a subject of the present invention as defined previously, mention may be made more particularly of alkyl polyglycosides comprising a linear or branched and saturated or unsaturated aliphatic radical and comprising from 8 to 16 carbon atoms, such as octyl polyglucoside, decyl polyglucoside, undecylenyl polyglucoside, dodecyl polyglucoside, tetradecyl polyglucoside, hexadecyl polyglucoside or 1,12-dodecanediyl polyglucoside; ethoxylated hydrogenated castor oil derivatives, such as the product sold under the INCI name “PEG-40 hydrogenated castor oil”; polysorbates, such as Polysorbate 20, Polysorbate 40, Polysorbate 60, Polysorbate 70, Polysorbate 80 or Polysorbate 85; coconut kernel amides; or N-alkylamines.

As examples of thickening and/or gelling surfactants that may be combined with the extract of Halopteris scoparia in the cosmetic composition that is a subject of the present invention as defined previously, mention may be made of optionally alkoxylated alkyl polyglycoside fatty esters, for instance ethoxylated methylpolyglucoside esters, such as the PEG 120 methyl glucose trioleate and the PEG 120 methyl glucose dioleate sold, respectively, under the names Glutamate™ LT and Glutamate™ DOE120; alkoxylated fatty esters, such as the PEG 150 pentaerythrityl tetrastearate sold under the name Crothix™ DS53, the PEG 55 propylene glycol oleate sold under the name Antil™ 141; fatty-chain polyalkylene glycol carbamates, for instance the PPG-14 laureth isophoryl dicarbamate sold under the name Elfacos™ T211, the PPG-14 palmeth-60 hexyl dicarbamate sold under the name Elfacos™ GT2125.

As examples of thickening and/or gelling agents that may be combined with the extract of Halopteris scoparia in the cosmetic composition that is a subject of the present invention as defined previously, mention may be made of linear or branched or crosslinked polymers of polyelectrolyte type, such as the partially or totally salified homopolymer of acrylic acid, the partially or totally salified homopolymer of methacrylic acid, the partially or totally salified homopolymer of 2-methyl-[(1-oxo-2-propenyl)amino]-1-propanesulfonic acid (AMPS), copolymers of acrylic acid and AMPS, copolymers of acrylamide and AMPS, copolymers of vinylpyrrolidone and AMPS, copolymers of AMPS and (2-hydroxyethyl) acrylate, copolymers of AMPS and (2-hydroxyethyl) methacrylate, copolymers of AMPS and hydroxyethyl acrylamide, copolymers of AMPS and N,N-dimethylacrylamide, copolymers of AMPS and tris(hydroxymethyl) acrylamidomethane (THAM), copolymers of acrylic or methacrylic acid and (2-hydroxyethyl) acrylate, copolymers of acrylic or methacrylic acid and (2-hydroxyethyl) methacrylate, copolymers of acrylic or methacrylic acid and hydroxyethylacrylamide, copolymers of acrylic or methacrylic acid and THAM, copolymers of acrylic or methacrylic acid and N,N-dimethylacrylamide, terpolymers of acrylic or methacrylic acid, AMPS and (2-hydroxyethyl) acrylate, terpolymers of acrylic or methacrylic acid, AMPS and (2-hydroxyethyl) methacrylate, terpolymers of acrylic or methacrylic acid, AMPS and THAM, terpolymers of acrylic or methacrylic acid, AMPS and N,N-dimethylacrylamide, terpolymers of acrylic or methacrylic acid, AMPS and acrylamide, copolymers of acrylic acid or methacrylic acid and alkyl acrylates in which the carbon chain comprises between four and thirty carbon atoms, more particularly between ten and thirty carbon atoms, copolymers of AMPS and alkyl acrylates in which the carbon chain comprises between four and thirty carbon atoms and more particularly between ten and thirty carbon atoms, linear, branched or crosslinked terpolymers of at least one monomer bearing a strong acid function, which is free, partially salified or totally salified, with at least one neutral monomer, and at least one monomer of formula (XIII):

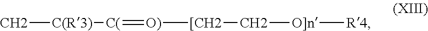

in which R′3 represents a hydrogen atom or a methyl radical, R′4 represents a linear or branched alkyl radical including from 8 to 30 carbon atoms and n′ represents a number greater than or equal to 1 and less than or equal to 50.

The linear or branched or crosslinked polymers of polyelectrolyte type, which may be combined with the extract of Halopteris scoparia in the cosmetic composition that is the subject of the present invention as defined previously, may be chosen from the products sold under the names Simulgel™ EG, Simulgel™ EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ Zen, Sepilife™ Nude, Aristoflex™ AVC, Aristoflex™ AVS, Aristoflex™ AVL, Aristoflex™ BLV, Aristoflex™ Silk, Aristoflex™ TAC, Aristoflex™ Velvet, Aristoflex™ A60, Aristoflex™ ECO T, Aristoflex™ PEA, Aristoflex™ PEA 70, Novemer™ EC-1, Novemer™ EC 2, Aristoflex™ HMB, Cosmedia™ SP, Flocare™ ET 25, Flocare™ ET 75, Flocare™ ET 26, Flocare™ ET 30, Flocare™ ET 58, Flocare™ PSD 30, Viscolam™ AT 64, Viscolam™ AT 100, Texique™ HE 10, Texique™ HE 20, Texique™ PQ 37.

As examples of thickening and/or gelling agents that may be combined with the extract of Halopteris scoparia in the cosmetic composition that is the subject of the present invention as defined previously, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickening and/or gelling agents that may be combined with the extract of Halopteris scoparia in the cosmetic composition that is the subject of the present invention as defined previously, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, or glucosaminoglycans.

As examples of thickening and/or gelling agents that may be combined with the extract of Halopteris scoparia in the cosmetic composition that is the subject of the present invention as defined previously, mention may be made of cellulose, cellulose derivatives such as methyl cellulose, ethyl cellulose, hydroxypropyl cellulose, silicates, starch, hydrophilic starch derivatives, or polyurethanes.

As examples of stabilizers that may be combined with the extract of Halopteris scoparia in the cosmetic composition that is the subject of the present invention as defined previously, mention may be made of microcrystalline waxes, and more particularly ozokerite, mineral salts such as sodium chloride or magnesium chloride, silicone polymers such as polysiloxane polyalkyl polyether copolymers.

As examples of solvents that may be combined with the extract of Halopteris scoparia in the cosmetic composition that is the subject of the present invention as defined previously, mention may be made of water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

As examples of thermal or mineral waters that may be combined with the extract of Halopteris scoparia in the cosmetic composition that is the subject of the present invention as defined previously, mention may be made of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-bains water, Allevard-les-Bains water, Digne water, Maizières water, Neyrac-les-Bains water, Lons-le-saunier water, Rochefort water, Saint Christau water, Les Fumades water and Tercis-les-Bains water.

As examples of hydrotropic agents that may be combined with the extract of Halopteris scoparia in the cosmetic composition that is the subject of the present invention as defined previously, mention may be made of xylenesulfonates, cumenesulfonates, hexyl polyglucoside, 2-ethylhexyl polyglucoside or n-heptyl polyglucoside.

As examples of emulsifying surfactants that may be combined with the extract of Halopteris scoparia in the cosmetic composition that is the subject of the present invention as defined previously, mention may be made of nonionic surfactants, anionic surfactants or cationic surfactants.

As examples of emulsifying nonionic surfactants that may be combined with the extract of Halopteris scoparia in the cosmetic composition that is the subject of the present invention as defined previously, mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucoside esters; alkyl polyglycosides including a linear or branched, saturated or unsaturated aliphatic radical, and including from 14 to 36 carbon atoms, for instance tetradecyl polyglucoside, hexyldecyl polyglucoside, octadecyl polyglucoside, hexadecyl polyxyloside, octadecyl polyxyloside, eicosyl polyglucoside, dodecosyl polyglucoside, 2-octyldodecyl polyxyloside, 12-hydroxystearyl polyglucoside; compositions of linear or branched, saturated or unsaturated fatty alcohols including from 14 to 36 carbon atoms and of alkyl polyglycosides as described previously, for example the compositions sold under the names Montanov™ 68, Montanov™ 14, Montanov™ 82, Montanov™ 202, Montanov™ S, Montanov™ WO18, Montanov™ L, Fluidanov™ 20X and Easynov™.

As examples of emulsifying anionic surfactants that may be combined with the extract of Halopteris scoparia in the cosmetic composition that is the subject of the present invention as defined previously, mention may be made of glyceryl stearate citrate, cetearyl sulfate, soaps, such as sodium stearate or triethanolammonium stearate, or N-acylamino acid derivatives which are salified, for example stearoyl glutamate.

As examples of emulsifying cationic surfactants that may be combined with the extract of Halopteris scoparia in the cosmetic composition that is the subject of the present invention as defined previously, mention may be made of amine oxides, quaternium-82 and the surfactants described in the patent application WO 96/00719 and mainly those the fatty chain of which comprises at least 16 carbon atoms.

As examples of opacifiers and/or nacreous agents that may be combined with the extract of Halopteris scoparia in the cosmetic composition that is the subject of the present invention as defined previously, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols including from 12 to 22 carbon atoms.

As examples of texturing agents that may be combined with the extract of Halopteris scoparia in the cosmetic composition that is the subject of the present invention as defined previously, mention may be made of N-acylamino acid derivatives, for instance lauroyl lysine sold under the name Aminohope™ LL, octenyl starch succinate sold under the name Dryflo™, myristyl polyglucoside sold under the name Montanov™ 14, cellulose fibres, cotton fibres, chitosan fibres, talc, sericite and mica.

As examples of deodorants that may be combined with the extract of Halopteris scoparia in the cosmetic composition that is the subject of the present invention as defined previously, mention may be made of alkali metal silicates; zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol; 1,3-propanediol; salicylic acid; sodium bicarbonate; cyclodextrins; metal zeolites; Triclosan™; aluminium bromohydrate, aluminium chlorohydrates, aluminium chloride, aluminium sulfate, aluminium zirconium chlorohydrates, aluminium zirconium trichlorohydrate, aluminium zirconium tetrachlorohydrate, aluminium zirconium pentachlorohydrate, aluminium zirconium octachlorohydrate, aluminium sulfate, sodium aluminium lactate, or complexes of aluminium chlorohydrate and of glycol, such as the aluminium chlorohydrate and propylene glycol complex, the aluminium dichlorohydrate and propylene glycol complex, the aluminium sesquichlorohydrate and propylene glycol complex, the aluminium chlorohydrate and polyethylene glycol complex, the aluminium dichlorohydrate and polyethylene glycol complex or the aluminium sesquichlorhydrate and polyethylene glycol complex.

As examples of oils that may be combined with the extract of Halopteris scoparia in the cosmetic composition that is the subject of the present invention as defined previously, mention may be made of the following compounds:
linear alkanes including from 11 to 19 carbon atoms, such as n-undecane, n-dodecane, n-tridecane, n-tetradecane, n-pentadecane, n-hexadecane, n-heptadecane, n-octadecane, n-nonadecane;
branched alkanes including from 7 to 40 carbon atoms, such as isododecane, isopentadecane, isohexadecane, isoheptadecane, isooctadecane, isononadecane or isoeicosane, or mixtures of certain thereof such as those mentioned hereinbelow and identified by their INCI name: C7-8 isoparaffin, C8-9 isoparaffin, C9-11 isoparaffin, C9-12 isoparaffin, C9-13 isoparaffin, C9-14 isoparaffin, C9-16 isoparaffin, C10-11 isoparaffin, C10-12 isoparaffin, C10-13 isoparaffin, C11-12 isoparaffin, C11-13 isoparaffin, C11-14 isoparaffin, C12-14 isoparaffin, C12-20 isoparaffin, C13-14 isoparaffin, C13-16 isoparaffin;
cycloalkanes optionally substituted with one or more linear or branched alkyl radicals;
white mineral oils, such as those sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (M1) which comprises, per 100% of its mass, a mass proportion of branched alkanes of greater than or equal to 90% and less than or equal to 100%; a mass proportion of linear alkanes of greater than or equal to 0% and less than or equal to 9%, and more particularly less than 5%, and a mass proportion of cycloalkanes of greater than or equal to 0% and less than or equal to 1%, for example the mixtures sold under the name Emogreen™ L15 or Emogreen™ L19;
mixtures of linear alkanes such as the mixture of n-undecane and n-tridecane such as the product sold under the brand name Cetiol Ultimate™;
mixtures of linear alkanes such as the mixture of n-dodecane and n-tetradecane sold under the brand name Vegelight™ 12-14;
fatty alcohol ethers of formula (II):
Z1-O—Z2 (II), in which Z1 and Z2, which may be identical or different, represent a linear or branched alkyl radical including from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (III):

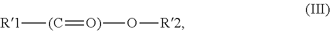

in which R′1-(C═O) represents a saturated or unsaturated, linear or branched acyl radical including from 8 to 24 carbon atoms, and R′2 represents, independently of R′1, a saturated or unsaturated, linear or branched hydrocarbon-based chain including from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (IV) and of formula (V):

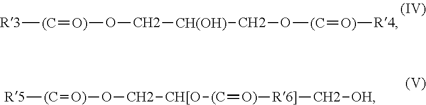

in which R′3-(C═O), R′4-(C═O), R′5-(C═O), R′6-(C═O), which may be identical or different, represent a saturated or unsaturated, linear or branched acyl group including from 8 to 24 carbon atoms;
triesters of fatty acids and of glycerol of formula (VI):

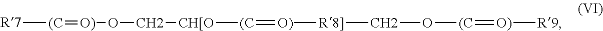

in which R′7-(C═O), R′8-(C═O) and R′9-(C═O), which may be identical or different, represent a saturated or unsaturated, linear or branched acyl group including from 8 to 24 carbon atoms;
plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, and oils derived from flowers or vegetables;
ethoxylated plant oils.

In the present patent application, the term “oils” refers to compounds and/or mixtures of compounds which are water-insoluble, and which have a liquid appearance at a temperature of 25° C.

As examples of waxes that may be combined with the extract of Halopteris scoparia in the cosmetic composition that is the subject of the present invention as defined previously, mention may be made of beeswax, carnauba wax, candelilla wax, ouricury wax, Japan wax, cork fibre wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” refers to compounds and/or mixtures of compounds which are water-insoluble, and which have a solid appearance at a temperature of greater than or equal to 45° C.

As examples of active principles that may be combined with the extract of Halopteris scoparia in the cosmetic composition that is the subject of the present invention as defined previously, mention may be made of vitamins and derivatives thereof, in particular esters thereof, such as retinol (vitamin A) and esters thereof (for example retinyl palmitate), ascorbic acid (vitamin C) and esters thereof, sugar derivatives of ascorbic acid (such as ascorbyl glucoside), tocopherol (vitamin E) and esters thereof (such as tocopheryl acetate), vitamins B3 or B10 (niacinamide and derivatives thereof); compounds with a lightening or depigmenting action on the skin, such as @-undecylenoyl phenylalanine sold under the name Sepiwhite™ MSH or Sepicalm™ VG, the glycerol mono- and/or diester of @-undecylenoyl phenylalanine, @-undecylenoyl dipeptides, arbutin, kojic acid, hydroquinone; compounds with a soothing action, including Sepicalm™ S, allantoin and bisabolol; anti-inflammatory agents; compounds with a moisturizing action, such as urea, hydroxyureas, glycerol, polyglycerols, glyceryl glucoside, diglyceryl glucoside, polyglyceryl glucosides, xylityl poglucoside; polyphenol-rich plant extracts such as grape extracts, pine extracts, wine extracts, olive extracts; compounds with a slimming or lipolytic action, such as caffeine or derivatives thereof, Adiposlim™, Adipoless™, fucoxanthin; N-acyl proteins; N-acyl peptides such as Matrixil™; N-acylamino acids; partial hydrolysates of N-acyl proteins; amino acids; peptides; total hydrolysates of proteins; soy extracts, for example Raffermin™; wheat extracts, for example Tensine™ or Gliadine™; plant extracts, such as plant extracts rich in tannins, plant extracts rich in isoflavones or plant extracts rich in terpenes; extracts of freshwater or marine algae; extracts of marine plants; marine extracts in general such as corals; essential waxes; bacterial extracts; ceramides; phospholipids; compounds with an antimicrobial or purifying action, such as Lipacide™ C8G, Lipacide™ UG, Sepicontrol™ A5; Octopirox™ or Sensiva™ SC50; compounds with an energizing or stimulating property, such as Physiogenyl™, panthenol and derivatives thereof such as Sepicap™ MP; anti-ageing active principles such as Sepilift™ DPHP, Lipacide™ PVB, Sepivinol™, Sepivitol™, Manoliva™, Phyto-Age™, Timecode™; Survicode™; anti-photo-ageing active principles; active principles for protecting the integrity of the dermo-epidermal junction; active principles for increasing the synthesis of components of the extracellular matrix such as collagen, elastins, glycosaminoglycans; active principles that act favourably on chemical cell communication such as cytokines or physical cell communication such as integrins; active ingredients that create a feeling of “heating” on the skin such as activators of skin microcirculation (such as nicotinic acid derivatives) or products that create a feeling of “freshness” on the skin (such as menthol and derivatives); active agents for improving cutaneous microcirculation, for example venotonics; draining active agents; active agents for decongesting purposes such as extracts of Ginkgo biloba, ivy, chestnut, bamboo, ruscus, butcher's broom, Centella asiatica, fucus, rosemary, willow; agents for tanning or browning the skin, for example dihydroxyacetone (DHA), erythrulose, mesotartaric aldehyde, glutaraldehyde, glyceraldehyde, alloxan, ninhydrin, plant extracts for example extracts of red wood of the genus Pterocarpus and of the genus Baphia such as Pteropcarpus santalinus, Pterocarpus osun, Pterocarpus soyauxii, Pterocarpus erinaceus, Pterocarpus indicus or Baphia nitida such as those described in European patent application EP 0 971 683; agents known for their action in facilitating and/or accelerating tanning and/or browning of human skin, and/or for their action in colouring human skin, for example carotenoids (and more particularly β-carotene and γ-carotene), the product sold under the brand name “carrot oil” (INCI name: Daucus carrota, Helianthus annuus sunflower oil) by Provital, which contain carotenoids, vitamin E, and vitamin K; tyrosine and/or derivatives thereof, known for their effect on the acceleration of the tanning of human skin in combination with exposure to ultraviolet radiation, for example the product sold under the brand name SunTan Accelerator™ by Provital, which contains tyrosine and riboflavins (vitamin B), the complex of tyrosine and of tyrosinase sold under the brand name Zymo Tan Complex by Zymo Line, the product sold under the brand name MelanoBronze™ (INCI name: Acetyl Tyrosine, Monk's pepper extract (Vitex agnus-castus)) by the company Mibelle, which contains acetyl tyrosine, the product sold under the brand name Unipertan VEG-24/242/2002 (INCI name:

Butylene Glycol and Acetyl Tyrosine and Hydrolysed Vegetable Protein and Adenosine Triphosphate) by the company Unipex, the product sold under the brand name Try-Excell™ (INCI name: Oleoyl tyrosine and Luffa cylindrica (seed) oil and oleic acid) by the company Sederma, which contains extracts of marrow seeds (or loofah oil), the product sold under the brand name Actibronze™ (INCI name: Hydrolysed wheat protein and acetyl tyrosine and copper gluconate) by the company Alban Muller, the product sold under the brand name Tyrostan™ (INCI name: Potassium caproyl tyrosine) by the company Synerga, the product sold under the brand name Tyrosinol (INCI name: Sorbitan Isostearate, Glyceryl Oleate, Caproyl Tyrosine) by the company Synerga, the product sold under the brand name InstaBronze™ (INCI name: Dihydroxyacetone and Acetyl Tyrosine and Copper Gluconate) by the company Alban Muller, the product sold under the brand name Tyrosilane (INCI name: Methylsilanol and Acetyl Tyrosine) by the company Exymol; peptides known for their effect in activating melanogenesis, for example the product sold under the brand name Bronzing SF Peptide Powder (INCI name: Dextran and Octapeptide-5) by the company Infinitec Activos, the product sold under the brand name Melitane (INCI name: Glycerin and aqua and dextran and acetyl hexapeptide-1) comprising acetyl hexapeptide-1 known for its alpha-MSH-agonist action, the product sold under the brand name Melatimes Solutions™ (INCI name: Butylene glycol, Palmitoyl Tripeptide-40) by the company Lipotec, sugars and sugar derivatives, for example the product sold under the brand name Tanositol™ (INCI name: inositol) by the company Provital, the product sold under the brand name Thalitan™ (or Phycosaccharide™ AG) by the company CODIF International (INCI name: Aqua and hydrolysed algin (Laminaria digitata) and magnesium sulfate and manganese sulfate) containing an oligosaccharide of marine origin (guluronic acid and mannuronic acid chelated with magnesium and manganese ions), the product sold under the brand name Melactiva™ (INCI name: Maltodextrin, Mucuna pruriens Seed Extract) by the company Alban Muller, compounds rich in flavonoids, for example the product sold under the brand name Biotanning (INCI name: Hydrolysed citrus Aurantium dulcis fruit extract) by the company Silab and known to be rich in lemon flavonoids (of the hesperidin type); agents intended for treating head hair and/or bodily hair, for example agents for protecting the melanocytes of the hair follicle, intended to protect said melanocytes against cytotoxic agents responsible for the senescence and/or apoptosis of said melanocytes, such as mimetics of DOPAchrome tautomerase activity, chosen from those described in the European patent application published under the number EP 1 515 688 A2, synthetic SOD mimetic molecules, for example manganese complexes, antioxidant compounds, for example cyclodextrin derivatives, siliceous compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

As examples of probiotics that may be combined with the extract of Halopteris scoparia in the cosmetic composition that is the subject of the present invention as defined previously, mention may be made of various strains of Saccharomyces cerevisiae, of Bacillus cereus var. toyoi, of Bacillus subtilis, alone or in combination with Bacillus licheniformis, or strains of Enteroccocus faecium. These strains of microorganisms are generally combined with a solid support, for example calcium carbonate, dextrose or sorbitol.

As examples of antioxidants that may be combined with the extract of Halopteris scoparia in the cosmetic composition that is the subject of the present invention as defined previously, mention may be made of EDTA and salts thereof, citric acid, tartaric acid, oxalic acid, BHA (butylhydroxyanisole), BHT (butylhydroxytoluene), tocopherol derivatives such as tocopheryl acetate, mixtures of antioxidant compounds such as Dissolvine™ GL 47S sold by the company AkzoNobel under the INCI name: Tetrasodium Glutamate Diacetate.

As examples of sunscreens that may be combined with the extract of Halopteris scoparia in the cosmetic composition that is the subject of the present invention as defined previously, mention may be made of all those appearing in the Cosmetic Directive 76/768/EEC, amended, Annex VII.

Among the organic sunscreens that may be combined with the extract of Halopteris scoparia in the cosmetic composition that is the subject of the present invention as defined previously, mention may be made of the family of benzoic acid derivatives such as para-aminobenzoic acids (PABA), notably monoglycerol esters of PABA, ethyl esters of N,N25 propoxy PABA, ethyl esters of N,N-diethoxy PABA, ethyl esters of N,N-dimethyl PABA, methyl esters of N,N-dimethyl PABA, butyl esters of N,N-dimethyl PABA; the family of anthranilic acid derivatives such as homomenthyl-N-acetyl anthranilate; the family of salicylic acid derivatives such as amyl salicylate, homomenthyl salicylate, ethylhexyl salicylate, phenyl salicylate, benzyl salicylate, p-isopropanolphenyl salicylate; the family of cinnamic acid derivatives such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy-2-ethylhexyl cinnamate), p-methoxy-2-ethoxyethyl cinnamate, p-methoxycyclohexyl cinnamate, ethyl-α-cyano-β-phenyl cinnamate, 2-ethylhexyl-α-cyano-β-phenyl cinnamate, glyceryl di-para-methoxymono-2-ethylhexanoyl cinnamate; the family of benzophenone derivatives such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone 5-sulfonate, 4-phenylbenzophenone, 2-ethylhexyl-4′-phenylbenzophenone 2-carboxylate, 2-hydroxy-4-n-octyloxybenzophenone, 4-hydroxy-3-carboxybenzophenone; 3-(4′-methylbenzylidene)-d,1-camphor, 3-(benzylidene)-d,1-camphor, camphorbenzalkonium methosulfate; urocanic acid, ethyl urocanate; the family of sulfonic acid derivatives such as 2-phenylbenzimidazole-5-sulfonic acid and salts thereof; the family of triazine derivatives such as hydroxyphenyltriazine, ethylhexyloxyhydroxyphenyl-4-methoxyphenyltriazine, 2,4,6-trianillino (p-carbo-2′-ethylhexyl-1′-oxy)-1,3,5-triazine, 4,4-((6-(((1,1-dimethylethyl)amino) carbonyl)phenyl)amino)-1,3,5-triazine-2,4-diyldiimino) bis(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methyphenyl)benzotriazole; dibenzalazine; dianisoylmethane, 4-methoxy-4″-t-butylbenzoylmethane; 5-(3,3-dimethyl-2-norbornylidene)-3-pentan-2-one; the family of the diphenylacrylate derivatives, such as 2-ethylhexyl 2-cyano-3,3-diphenyl-2-propenoate or ethyl 2-cyano-3,3-diphenyl-2-propenoate; or the family of polysiloxanes, such as benzylidene siloxane malonate.

Among the mineral sunscreens, also known as “mineral sunblocks”, that may be combined with the extract of Halopteris scoparia in the cosmetic composition that is the subject of the present invention as defined previously, mention may be made of titanium oxides, zinc oxides, cerium oxide, zirconium oxide, yellow, red or black iron oxides, and chromium oxides. These mineral sunblocks may or may not be micronized, may or may not have been subjected to surface treatments and may optionally be in the form of aqueous or oily predispersions.

A subject of the present invention is also a cosmetic care process comprising a step of topical application to the skin and/or to the scalp of a cosmetically effective amount of the extract of Halopteris scoparia as defined above or of the cosmetic composition containing same as defined above, for preventing and/or reducing and/or slowing down and/or stopping and/or attenuating the appearance of unsightly and/or uncomfortable and/or unpleasant manifestations of the skin and/or scalp in humans due to the secretion of sebum, for example chosen from the presence of closed comedones (whiteheads or microcysts) and/or open comedones (blackheads) and/or papules and/or pustules and/or nodules and/or scars and/or visibly dilated pores and/or the glistening or greasy or shiny appearance and/or the tacky sensation.

According to a particular aspect of the invention, the cosmetic care process also comprises an additional step, for example a step of washing the area of the skin and/or scalp to which the extract of Halopteris scoparia as defined above or the cosmetic composition containing same as defined above is applied topically.

A subject of the present invention is also an extract of Halopteris scoparia for its topical use in a therapeutic method aimed at reducing the amount of sebum, in particular excess sebum, produced by the skin and/or scalp in humans, in particular in a therapeutic method for preventing and/or treating skin pathologies associated with hyperproduction of sebum, such as acne, pathological hyperseborrhea and/or seborrheic eczema.

According to a particular aspect of the present invention, the skin pathologies associated with the hyperproduction of sebum are chosen from the group consisting of acne, pathological hyperseborrhea and/or seborrheic eczema.

According to a particular aspect of the present invention, the extract of Halopteris scoparia used in a therapeutic method aimed at reducing the amount of sebum produced by the skin and/or the scalp is as defined above.

According to a particular aspect of the present invention, the extract of Halopteris scoparia is present in a pharmaceutical composition intended for topical application, also comprising at least one pharmaceutically acceptable ingredient. In particular, the extract of Halopteris scoparia is present as active principle in said pharmaceutical composition for topical use in an effective amount, in particular in a content of from 0.1% by mass to 30% by mass, in particular from 1% by mass to 10% by mass, relative to the total weight of the pharmaceutical composition.

A subject of the present invention is also a pharmaceutical composition for topical use comprising as active principle an effective amount of an extract of Halopteris scoparia and at least one pharmaceutically acceptable ingredient, for use in a therapeutic method aimed at reducing the amount of sebum, in particular excess sebum, produced by the skin and/or scalp in humans, in particular in a therapeutic method for preventing and/or treating skin pathologies associated with hyperproduction of sebum, in particular chosen from the group consisting of acne, pathological hyperseborrhea and/or seborrheic eczema.

The effective amount of an extract of Halopteris scoparia included in the pharmaceutical composition reduces the amount of sebum, in particular excess sebum, produced by the skin and/or scalp in humans; in particular, the anti-lipase activity of the treated skin is greater than 50% compared to the control, and/or the production of lipid by the sebocytes of the treated skin is inhibited, and/or the formation of the biofilm of pathogenic bacteria is decreased or slowed down or inhibited.

According to a more particular aspect of the present invention, said pharmaceutical composition comprises, per 100% by mass:
from 0.1% by mass to 70% by mass of the extract of Halopteris scoparia as defined above and
from 30% by mass to 99.9% by mass of at least one pharmaceutically acceptable technological additive.

According to a more particular aspect of the present invention, said pharmaceutical composition comprises, per 100% by mass:
from 0.3% by mass to 60% by mass of the extract of Halopteris scoparia as defined above and
from 40% by mass to 99.7% by mass of at least one pharmaceutically acceptable technological additive.

According to a more particular aspect of the present invention, said pharmaceutical composition comprises, per 100% by mass:
from 0.5% by mass to 50% by mass of the extract of Halopteris scoparia as defined above and
from 50% by mass to 99.5% by mass of at least one pharmaceutically acceptable technological additive.

According to a more particular aspect of the present invention, said pharmaceutical composition comprises, per 100% by mass:
from 1.0% by mass to 40% by mass of the extract of Halopteris scoparia as defined above and
from 60% by mass to 99.0% by mass of at least one pharmaceutically acceptable technological additive.

According to a more particular aspect of the present invention, said pharmaceutical composition comprises, per 100% by mass:
from 2.0% by mass to 30% by mass of the extract of Halopteris scoparia as defined above and
from 70% by mass to 98.0% by mass of at least one pharmaceutically acceptable technological additive.

According to a more particular aspect of the present invention, said pharmaceutical composition comprises, per 100% by mass:
from 3.0% by mass to 20% by mass of the extract of Halopteris scoparia as defined above and
from 80% by mass to 97.0% by mass of at least one pharmaceutically acceptable technological additive.

According to a more particular aspect of the present invention, said pharmaceutical composition comprises, per 100% by mass:
from 5.0% by mass to 10% by mass of the extract of Halopteris scoparia as defined above and
from 90% by mass to 95.0% by mass of at least one pharmaceutically acceptable technological additive.

In the context of the invention, the pharmaceutically acceptable ingredient is a technological additive usually used in the field of formulations for topical use.

The term “technological additive” denotes any chemical substance or any chemical composition whose technical function is to enable and/or facilitate the mixing of the various constituents of said pharmaceutical composition, to facilitate and/or to optimize the physical properties of said pharmaceutical composition, such as to facilitate and/or optimize its flow, its stability and its incorporation in a pharmaceutical formulation, and which are able to meet the conditions required by the regulations in force for the marketing of a pharmaceutical formulation.

The pharmaceutical composition for topical use as defined previously may also comprise one or more technological additives chosen from solvents and co-solvents, or agents for improving skin penetration.

As examples of solvents and co-solvents optionally present in the pharmaceutical compositions for topical use that are the subject of the present invention, mention may be made of water, organic solvents, for instance glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate and benzyl alcohol.

As examples of agents for improving the skin penetration that may be present in the pharmaceutical compositions for topical use that are the subject of the present invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono-n-butyl ether, diethylene glycol monoethyl ether (or Transcutol-P), fatty acids such as oleic acid, fatty acid esters of glycerol, for instance glyceryl behenate, glyceryl palmitostearate, behenoyl macroglycerides, polyoxyethylene-2-stearyl ether, polyoxyethylene-2-oleyl ethers, terpenes, for instance D-limonene, and essential oils, for instance essential oil of Eucalyptus.

In general, the pharmaceutical composition according to the invention may comprise excipients and/or active principles commonly used in the field of formulations for topical, in particular pharmaceutical or dermopharmaceutical, use.

As examples of active agents that may be combined with the pharmaceutical composition according to the invention, mention may be made of substances or compositions which provide a beneficial effect to the human subject.

These active agents may be, for example, antibodies, analgesics, anti-inflammatory agents, cytokines, cytoxins, growth factors, hormones, lipids, oligonucleotides, polymers, polysaccharides, polypeptides, protease inhibitors, vitamins, insect repellents, antibiotics or antifungal agents.

As examples of analgesic and anti-inflammatory agents that may be combined with the pharmaceutical composition according to the invention, mention may be made of acetaminophen, aspirin, salicylic acid, methyl salicylate, choline salicylate, glycol salicylate, 1-menthol, camphor, mefenamic acid, fluphenamic acid, indomethacin, protizidic acid, fentiazac, tolmetin, tiaprofenic acid, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone.

As examples of antiseptic agents that may be combined with the pharmaceutical composition according to the invention, mention may be made of cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride.

As examples of anti-insecticidal agents that may be combined with the pharmaceutical composition according to the invention, mention may be made of trichlorfone, triflumerone, fenthion, bendiocarb, cyromazine, dislubenzurone, dicyclanil, fluazurone, amitraz, deltamethrin, cypermethrin, chlorfenbinphose, flumethrin, ivermectin, abermectin, avermectin, doramectin, moxidectin, zeti-cypermethrin, diazinone, spinosad, imidacloprid, nitenpyran, pyriproxysene, sipronil, cythioate, lufenurone, selamectin, milbemycin oxime, chlorpyrifose, coumaphose, propetamphose, alpha-cypermethrin, cypermethrin high-cis, ivermectin, diflubenzurone, cyclodiene, carbamate and benzoyl urea.

As examples of antimicrobial agents that may be combined with the pharmaceutical composition according to the invention, mention may be made of sulfonamides, aminoglycosides, for instance neomycin, tobramycin, gentamycin, amikacin, kanamycin, spectinomycin, paromomycin, netilmicin, polypeptides, cephalosporins, oxazolidinones, for instance ciprofloxacin, levofloxacin and ofloxacin.

As examples of active agents that may be combined with the pharmaceutical composition according to the invention, mention may be made of vitamin E, Coenzyme Q10, L-carnitine, choline, folic acid, magnesium and salts thereof, caprylic acid, linoleic acid, lauric acid, taurine, vitamin C, vitamin A, and group B vitamins.

Application to the skin of the extract of Halopteris scoparia as defined above or of the pharmaceutical composition containing same makes it possible:
to limit the formation of the biofilm of opportunistic pathogenic bacteria, for instance Staphylococcus aureus, without detrimentally affecting the presence of commensal bacteria such as Staphylococcus epidermidis,
to protect sebocytes from the overproduction of lipids, and
to reinforce an antilipase activity of the skin or of the scalp;
   effects which are responsible for the development of sebum on the skin and the scalp, and consequently of the associated unsightly effects.

### EXAMPLES

The examples that follow illustrate the invention without, however, limiting it.

Dry Halopteris scoparia alga is ground and then placed in contact with a solvent composed of 90% water and 10% of a mixture of diols composed of 5% 1,3-propanediol (CAS 504-63-2) and 5% 1,2-pentanediol (CAS 5343-92-0). The alga/solvent mass ratio is 10/90. The mixture is stirred for 2 hours at 20° C. The plant matter is then separated from the solvent by centrifugation and final filtration at 0.2 μm. The solvent is conserved to obtain a final liquid extract having a dry matter content of between 0.5% and 4%. In the example of the invention, the composition of the final extract obtained is composed of 88.80% by mass of water, 4.90% by mass of 1,3-propanediol, 4.90% by mass of 1,2-pentanediol and 1.40% by mass of dry matter. The Halopteris scoparia extract is incorporated into formulations at the mass contents indicated for the following tests.

### A. Decrease in Sebum and Rate of Regreasing In Vivo

### Principle of the Method:

Women with combination/greasy to greasy skin and sebum levels between 140 and 190 μg/cm² were recruited to evaluate the seboregulatory effect of Halopteris scoparia extract versus placebo. They applied the products to their face twice a day for 28 days, measured on DO (before application of the products) and on D28. The sebum level was measured with a sebumeter.

### Formulations Tested:

TABLE 1

| | | Formula containing |
|---|---|---|
| | | the extract of |
| | | Halopteris |
| Ingredients | Placebo | scoparia 1% |
| Water | qs 100% | |
| Glycerol | 1% | |
| Montanov L | 1% | |
| (C14-22 alcohols & C12-20 alkyl | | |
| glucoside) | | |
| Simulsol 165 | 1% | |
| (PEG-100 stearate & glyceryl stearate) | | |
| Triglyceride 5545 | 3% | |
| (caprylic/caprylic triglycerides) | | |
| Montanov L | 7% | |
| (C14-22 alcohols & C12-20 alkyl | | |
| glucoside) | | |
| Sepinov EMT10 | 0.5% | |
| (Hydroxyethyl acrylate/sodium | | |
| acryloyldimethyl taurate copolymer) | | |
| Sepimax Zen | 1% | |
| (Polyacrylate crosspolymer-6) | | |
| Extract of Halopteris scoparia (water | — | 1% |

| & pentylene glycol & propanediol | |
|---|---|
| & Halopteris scoparia extract) | |
| Euxyl PE9010 | 1% |
| (phenoxyethanol & ehtylhexylglycerol) | |
| Sensiva PA40 | 0.5% |
| (phenylpropanol & caprylyl glycol & | |
| propanediol & tocopherol) | |
| Neutrophil fragrance | 0.04% |

The sebum level on the skin was evaluated with a Sebumeter® SM 815 (Courage & Khazaka).

It uses a cassette equipped with a transparent film sensitive to absorbed lipids (sebum) and a photometric sensor. The film is applied to the measurement area for 30 seconds. Its transparency increases in proportion to the amount of sebum in the hydrolipid film with which it is in contact, which makes it possible to calculate a sebum level per unit area (in μg/cm²).

To assess the rate of skin regreasing, the anterior forehead area was defatted with an ethanol/water (70/30) mixture until a sebum value of ≤50 g/cm² was obtained, measured using a Sebumeter® SM 815. The sebum level was then measured again 30 minutes after defatting and the regreasing rate was calculated using the following formula: (sebum level 30 minutes after defatting-sebum level before defatting)/30 minutes.

### Statistical Elements:

The values obtained are expressed as a mean±SEM (standard error of the mean).

A percentage effect of D28 versus DO was calculated:

$$\% Effect = \frac{Mean ( D 28 ) - Mean ( D 0 )}{Mean ( D 0 )} \times 100 \qquad Math 1$$

The intra-group analysis was performed with a paired Student t-test if the normality hypothesis was validated, otherwise with a Wilcoxon rank test.

The inter-group analysis was performed on the sebum levels (D28-DO) and on the regreasing rates at D28 with an unpaired Student t-test if the normality hypothesis was validated, otherwise with a Wilcoxon rank test.

Changes were considered statistically significant when the p-value was <0.05.

### Results:

Table 1 below presents the results on the sebum level at D0 and D28 (μg/cm²). The values are expressed as mean±SEM.

TABLE 1

| | D 0 | D 28 | D 28 − D 0 | Statistics |
|---|---|---|---|---|
| Placebo | 170.8 ± 2.8 | 136.9 ± 6.8 | −33.9 ± 7.9 | p < 0.001 vs |
| | | | (−20%) | D 0 |
| Halopteris | 169.4 ± 3.7 | 111.2 ± 9.5 | −58.2 ± 9.4 | p < 0.001 vs |
| scoparia 1% | | | (−34%) | D 0 |
| | | | | p = 0.044 |
| | | | | vs placebo |

Table 2 below presents the results on the regreasing rate at D0 and D28 (μg/cm²). The values are expressed as mean±SEM.

TABLE 2

| | D 0 | D 28 | D 28 − D 0 | Statistics |
|---|---|---|---|---|
| Placebo | 87.9 ± 7.8 | 71.0 ± 7.9 | −17.0 ± 9.5 (−19%) | p = 0.09 vs |
| | | | | D 0 |
| Halopteris | 72.8 ± 7.0 | 40.7 ± 4.2 | −32.1 ± 8.8 (−44%) | p = 0.002 vs |
| scoparia 1% | | | | D 0 |
| | | | | p = 0.003 vs |
| | | | | placebo |

### B. Production of Sebum by Sebocytes Under Inflammatory Conditions

### Principle of the Method:

Caucasian sebocytes derived from hiPSc (human induced pluripotent stem cells) technology are directly thawed in a 96-well plate, previously coated with fibronectin, and cultured for 3 days in a suitable culture medium supplemented with a complement allowing their maturation. They are then co-treated with the ingredients to be tested+arachidonic acid at 15 μM for 48 hours. Each condition is performed in quadriplicate (n=4).

The sebocytes are fixed, permeabilized and labelled with the BodiPy probe (lipids) and Hoechst 33342 (nuclei).

The cells are then imaged using an automated microscope (CellInsight CX5) at 20X and 24 fields are acquired per well. The fluorescence intensity is then quantified and normalized to the amount of nuclei.

### Statistical Elements:

The values obtained were expressed as mean±SD (standard deviation).

For each condition, a percent effect was calculated:

$$\% Effect = \frac{Mean ( X ) - Mean ( stressed cells )}{Mean ( stressed cells )} \times 100 \qquad Math 2$$

X: condition studied
Stressed cells: sebocytes treated with arachidonic acid
A percentage of protection was also calculated:

$$\% protection = \frac{Mean ( X ) - Mean ( stressed cells )}{Mean ( unstressed cells ) - Mean ( stressed cells )} \times 100 \qquad Math 3$$

X: condition studied
Unstressed cells: untreated sebocytes (control condition)
Stressed cells: sebocytes treated with arachidonic acid
Statistical analysis was performed using a Student's test with a significance level set at 5%.

### Results:

Table 3 below presents the results of the effect of the extract of Halopteris scoparia on arachidonic acid-induced lipid overproduction on Caucasian sebocytes from iPS.

TABLE 3

| | Mean ± SD | | % |
|---|---|---|---|
| | (arbitrary unit) | % effect | protection |
| Untreated cells | 70 ± 40 | — | — |
| (control) | | | |
| +arachidonic acid | 10944 ± 1984 | +15610%** vs | — |
| (15 μM) | | untreated cells | |
| +Halopteris | 5041 ± 4170 | −54%* vs | 54%** |
| scoparia 0.1% | | stressed cells | |

**p < 0.01

Analysis of the images obtained after BodiPy labelling shows that, after 48 h of incubation, arachidonic acid stimulates lipid production by the sebocytes (+15610%, p<0.01).

The extract of Halopteris scoparia reduces the arachidonic acid-induced lipid overproduction by 54% (p<0.01).

C. sebum Production by Sebocytes Induced by C. acnes Extracellular Vesicles (EVs)

### Principle of the Method:

Caucasian sebocytes derived from iPS (Phenocell) are thawed and seeded directly in a 48-well plate previously coated with fibronectin at 27 500 cells/well.

The cells are then incubated for 3 days at 37° C. under 5% CO₂ and then treated for 24 h with the Halopteris scoparia extract at 0.1%.

The cells are then placed in contact with the extracellular vesicles (EVs) derived from the virulent strain DSM1897 of C. acnes (26 μg/mL) for 48 hours.

BodiPy labelling is performed to study the influence of the EVs on lipid production.

### Statistical Elements:

The values obtained are expressed as mean±SD (standard deviation). For each condition, a percent effect was calculated:

$$\% Effect = \frac{Mean ( X ) - Mean ( stressed cells )}{Mean ( stressed cells )} \times 100 \qquad Math 2$$

X: condition studied
Stressed cells: sebocytes treated with the EVs
A percentage of protection was also calculated:

$$\% protection = \frac{Mean ( X ) - Mean ( stressed cells )}{Mean ( unstressed cells ) - Mean ( stressed cells )} \times 100 \qquad Math 3$$

X: condition studied
Unstressed cells: untreated sebocytes (control condition)
Stressed cells: sebocytes treated with the EVs
Statistical analysis was performed using a Student's test with a significance level set at 5%.

### Results:

Table 4 below presents the results of the effect of the Halopteris scoparia extract on lipid overproduction induced by the EVs of C. acnes on Caucasian sebocytes from iPS.

TABLE 4

| | Mean ± SD | | % |
|---|---|---|---|
| | (arbitrary unit) | % effect | protection |
| Untreated cells | 128 ± 36 | — | — |
| (control) | | | |
| +EVs DSM1897 (26 | 316 ± 48 | +146%** vs | — |
| μg/mL) | | untreated cells | |
| +Halopteris | 114 ± 43 | −64%** vs | 108%** |
| scoparia 0.1% | | stressed cells | |

**p < 0.01

Analysis of the images obtained after BodiPy labelling shows that after 48 h of incubation, the extracellular vesicles of C. acnes stimulate lipid production by the sebocytes (+146%, p<0.01).

The Halopteris scoparia extract inhibits the lipid overproduction induced by the EVs of C. acnes by 64% (p<0.01).

D. Harmlessness with Respect to the Cutaneous Microbiota

### Principle of the Method:

The test methodology is described below:

A co-culture (consortium) of the five main bacteria of the facial microbiota (Cutibacterium acnes, Staphylococcus epidermidis, Streptococcus mitis, Micrococcus luteus and Corynebacterium xerosis) is performed from wild-type bacterial isolates. The bacteria are in proportions representative of the microbiota of the cheek. The consortium is incubated under conditions mimicking those encountered on the skin (low pH, low temperature, low nutrient concentration) for 8 hours (maximum contact time of a cosmetic product).

After eight hours of contact with an extract of Halopteris scoparia, the bacterial concentrations are the same as in the control (consortium without any product), indicating that an extract of Halopteris scoparia has no significant negative impact on the main skin bacteria and can be considered “microbiota friendly”.

The following bacterial strains (commensal strains of the skin microbiota) were selected and used to produce a calibrated consortium:
Cutibacterium acnes, 58%
Staphylococcus epidermidis, 18%
Streptococcus sp., 14.6%
Micrococcus luteus, 4.4%
Corynebacterium xerosis, 5%

This consortium was placed in contact for 8 h with the extract of Halopteris scoparia 1% and the concentrations of each species were measured. Logarithmic reduction (LR) was calculated relative to the control (bacteria without Halopteris scoparia extract) for each bacterial species, such that:

$${LR}_{species X} = ( log CFU / mL for test product ) - ( log CFU / mL control ) \qquad Math 4$$

The impact of the extract was measured by establishing a score, which is equal to the addition of the LRs obtained for the five bacterial species:

$$Score = {LR}_{C . acnes} + {LR}_{S . epidermidis} + {LR}_{S . mitis} + {LR}_{M . luteus} + {LR}_{C . xerosis} \qquad Math 5$$

A logarithmic reduction of less than or equal to 1 (LR≤1) is not considered significant. Therefore, a score between +5 and −5 indicates a microbiota-neutral (microbiota-friendly) product.

### Results:

The logarithmic reductions obtained for each species were:

$${RL}_{C . acnes} = - 0.54$$

$${RL}_{S . epidermidis} = 0.35$$

$${RL}_{S . mitis} = 0.$$

$${RL}_{M . luteus} = - 0.32$$

$${RL}_{C . xerosis} = - 0.12$$

The score obtained for the extract of Halopteris scoparia according to this model is therefore −0.63, which means that there is no modification of the microbial consortium in the presence of the extract of H. scoparia. Since this score is between-5 and +5, the extract is considered microbiota-friendly.

### CONCLUSION

TABLE 5

| | Results |
|---|---|
| Clinical | Decrease in sebum and regreasing |
| | rate |
| Lipid production by sebocytes | Decrease in lipid production |
| Effects of C. acnes EVs on | Decrease in lipid production |
| lipid production | |
| Harmless with respect to the | No impact on commensal strains |
| cutaneous microbiota | of the cheek |

1. A method for preventing, reducing, slowing down, stopping, and/or attenuating a presence of closed comedones, open comedones, papules, pustules, nodules, scars, visibly dilated pores, a glistening or greasy or shiny appearance, and/or a tacky sensation of skin and/or a scalp of humans due to secretion of sebum from essentially healthy skin, the method comprising:
topically applying an extract of Halopteris scoparia as a cosmetic active principle to the skin and/or the scalp of a subject in need thereof.

2. The method according to claim 1, wherein the extract of Halopteris scoparia comprises, per 100% of its mass:
from 80% by mass to 95% by mass of water;
from 4.5% by mass to 15% by mass of an organic solvent selected from the group consisting of 1,2-propanediol, 1,3-propanediol, 1,4-butanediol, 1,3-butanediol, 1,2-butanediol, 1,2-pentanediol, 2-methyl-2,4-pentanediol, 1,6-hexanediol, 1,8-octanediol, and a mixture of these compounds; and
from 0.5% by mass to 5.0% by mass of dry matter.

3. The method according to claim 2, wherein the organic solvent is a mixture of 1,3-propanediol and 1,2-pentanediol.

4. The method according to claim 2, wherein the organic solvent comprises, per 100% of its mass, a mixture of two diols different from each other in equal mass proportions.

5. The method according to claim 1, wherein the extract of Halopteris scoparia is obtained via a process comprising:
a) drying and grinding Halopteris scoparia to obtain a biomass;
b) bringing the biomass obtained in step a) into contact with an organic solvent so as to obtain a mixture, the mixture comprising, per 100% of its mass, from 5% by mass to 15% by mass of the biomass and from 85% by mass to 95% by mass of the organic solvent;
c) stirring the mixture obtained in step b) for a period of between 1 hour and 6 hours at a temperature of between 15° C. and 35° C.; and
d) oseparating the biomass from the mixture obtained at the end of step c) to isolate a liquid phase which corresponds to the extract of Halopteris scoparia.

6. The method according to claim 1, wherein the extract of Halopteris scoparia is applied to unsightly, uncomfortable, and/or unpleasant manifestations of the skin and/or the scalp in humans due to the secretion of sebum, wherein the manifestations are selected from the group consisting of closed comedones, open comedones, papules, pustules, nodules, scars, visibly dilated pores, the glistening or greasy or shiny appearance, the tacky sensation, and combinations thereof.

7. The method according to claim 1, wherein the extract of Halopteris scoparia is topically applied in a cosmetic composition, wherein the cosmetic composition comprises at least one cosmetically acceptable ingredient and the extract of Halopteris scoparia, wherein the extract of Halopteris scoparia is present in the cosmetic composition in an amount from 0.01% by mass to 5.00% by mass relative to a total weight of the cosmetic composition.

8. A method of obtaining an extract of Halopteris scoparia in a form suitable for topical application for its therapeutic use for preventing and/or treating skin pathologies associated with hyperproduction of sebum, the method comprising:
a) drying and grinding Halopteris scoparia to obtain a biomass;
b) bringing the biomass obtained in step a) into contact with an organic solvent so as to obtain a mixture, the mixture comprising, per 100% of its mass, from 5% by mass to 15% by mass of the biomass and from 85% by mass to 95% by mass of the organic solvent;
c) stirring the mixture obtained in step b) for a period of between 1 hour and 6 hours at a temperature of between 15° C. and 35° C.; and
d) separating the biomass from the mixture obtained at the end of step c) to isolate a liquid phase which corresponds to the extract of Halopteris scoparia.

9. A composition comprising the extract of Halopteris scoparia obtained according to the method of claim 8, wherein the extract of Halopteris scoparia comprises, per 100% of its mass:
from 80% by mass to 95% by mass of water;
from 4.5% by mass to 15% by mass of an organic solvent selected from the group consisting of 1,2-propanediol, 1,3-propanediol, 1,4-butanediol, 1,3-butanediol, 1,2-butanediol, 1,2-pentanediol, 2-methyl-2,4-pentanediol, 1,6-hexanediol, 1,8-octanediol, and a mixture of these compounds;
from 0.5% by mass to 5.0% by mass of dry matter.

10. A pharmaceutical composition comprising at least one pharmaceutically acceptable ingredient and the extract of Halopteris scoparia obtained according to the method of claim 8, wherein the extract of Halopteris scoparia is present in the pharmaceutical composition in an amount from 0.1% by mass to 30% by mass relative to a total weight of the pharmaceutical composition.

11. A pharmaceutical composition comprising as active principle an effective amount of the extract of Halopteris scoparia obtained according to the method of claim 8 and at least one pharmaceutically acceptable ingredient, wherein the pharmaceutical composition is in a form suitable for topical application for its therapeutic use for preventing and/or treating skin pathologies associated with hyperproduction of sebum.

12. The method according to claim 6, wherein the closed comedones are whiteheads or microcysts.

13. The method according to claim 6, wherein the open comedones are blackheads.

14. The method according to claim 7, wherein the extract of Halopteris scoparia is present at 1% by mass relative to the total weight of the cosmetic composition.

15. The method according to claim 8, wherein the extract is capable of preventing and/or treating acne, pathological hyperseborrhea and/or seborrheic eczema.

16. The pharmaceutical composition according to claim 11, wherein the pharmaceutical composition is capable of preventing and/or treating acne, pathological hyperseborrhea, and/or seborrheic eczema.
